(19) United States
(12) Patent Application Publication
(10) Pub. No.: US 2015/0297786 A1
(43) Pub. Date: Oct. 22, 2015

(54) Fenestrated Hemostatic Patch

(71) Applicant: Ethicon, Inc., Somerville, NJ (US)

(72) Inventors: Jerome Riebman, Basking Ridge, NJ (US); Jeffrey Hamond, Bernardsville, NJ (US); Kevin Weadock, Hillsborough, NJ (US); John P. Matonick, Warren, NJ (US)

(21) Appl. No.: 14/791,718

(22) Filed: Jul. 6, 2015

Related U.S. Application Data
(62) Division of application No. 13/625,968, filed on Sep. 25, 2012.
(60) Provisional application No. 61/540,666, filed on Sep. 29, 2011.

(51) Int. Cl. A61L 26/00

(52) U.S. Cl. CPC A61L 26/0042; A61L 26/0047; A61L 26/0066; A61L 26/009; A61L 2300/254; A61L 2300/252; A61L 2300/418; A61L 2300/608; A61L 2400/04

(57) ABSTRACT

A hemostat comprises a non-porous patch made of a bio-resorbable tissue compatible material, said patch having a tissue-facing surface and a top surface; an optional hemostatic agent that is disposed on said tissue-facing surface or optionally dispersed throughout said patch or optionally as a separate carrier layer, and at least one aperture or slit that penetrates said patch at least partially from said tissue-facing surface to said top surface.

### FIELD OF THE INVENTION

The present invention relates generally to agents and devices for promoting hemostasis and, more particularly, to hemostatic patches comprising biological factors disposed on or within absorbable scaffolds.

### BACKGROUND OF THE INVENTION

Prolonged or uncontrolled bleeding following trauma or after surgery of the cardiovascular system, liver, kidney, spleen, and other organs is a serious complication associated with significant morbidity, mortality, and cost of care. Meticulous surgical technique is essential for primary hemostasis, but when conventional techniques such as compression, ligation, clipping, and electrocautery are impractical or ineffective, topical hemostatic agents are indispensable and relied upon to help control blood loss.

The control of bleeding is essential and critical in surgical procedures to minimize blood loss, to reduce post-surgical complications, and to shorten the duration of the surgery in the operating room. In an effort to address the above-described problems, materials have been developed for controlling excessive bleeding. Many approved and investigational topical hemostats have been described in the literature, including oxidized regenerated cellulose (ORC), collagen, gelatin, fibrinogen, thrombin, polymers, and inorganic compounds. Some of these rely on physical means for passive coagulation (ORC, gelatin, collagen) while others provide biologically active components of the clotting cascade (thrombin and fibrin sealants). The latest generation of topical hemostats combines both active and passive mechanisms of action.

Topical Absorbable Hemostats (TAHs) are widely used in surgical applications. TAHs encompass products based on oxidized cellulose (OC), oxidized regenerated cellulose (ORC), gelatin, collagen, chitin, chitosan, etc. To improve the hemostatic performance, scaffolds based on the above materials can be combined with biologically-derived clotting factors, such as thrombin and fibrinogen.

Some currently utilized hemostatic wound dressings include knitted or non-woven fabrics comprising carboxylic-oxidized cellulose. Examples of such hemostatic wound dressings commercially available include Surgicel® absorbable hemostat; Surgicel Nu-Knit® absorbable hemostat; and Surgicel® Fibrillar absorbable hemostat; all available from Johnson & Johnson Wound Management Worldwide, a division of Ethicon, Inc., Somerville, N.J., a Johnson & Johnson Company.

Other currently utilized hemostatic wound dressings comprise gelatin or collagen, for example Surgiflo® absorbable hemostat available from Johnson & Johnson Wound Management Worldwide, a division of Ethicon, Inc., Somerville, N.J.

Some currently utilized hemostatic wound dressings and sealants comprise blood clotting factors such as thrombin and/or fibrinogen, which can be animal derived or human origin blood clotting factors. Such biologic factors-containing hemostatic wound dressings and sealants can be in the form of liquid sprays, such as sealant EVICEL® Fibrin Sealant (Human), available from Johnson & Johnson Wound Management Worldwide, a division of Ethicon, Inc., Somerville, N.J.

Some currently utilized hemostatic wound dressings and sealants comprise blood clotting factors such as thrombin and/or fibrinogen which are dispersed in a semi-solid carrier, such as gelatin, or in or on a solid substrate. TachoSil® is an absorbable fibrin sealant patch where human fibrinogen and human thrombin are coated onto an equine collagen sponge; TachoSil® is available from Baxter International, Deerfield, Ill.

Decreasing the time to achieve hemostasis has great clinical significance—to save blood loss and speed up the procedure. The majority of current products on the market in case of mild to moderate bleeding achieve hemostasis in a time frame from about 4 to 8 minutes. In addition, many products do not have ideal handling characteristics as they wrinkle and fold during surgical procedures especially in the presence of blood or other fluids. A medical needs remains for hemostatic devices that have better mechanical properties, particularly for use in laparoscopic procedures. Finally, some products when used in multiple layers or those in particulate form may disintegrate or their parts may migrate during the application process. There is a clear medical need to achieve faster hemostasis to reduce blood loss during surgery as well as a desire to provide improved handling performance and an improved ability to stay in place after application.

U.S. Pat. No. 7,279,177 B2 assigned to Ethicon is directed to a hemostatic wound dressing that utilizes a fibrous, fabric substrate made from carboxylic-oxidized cellulose and containing a first surface and a second surface opposing the first surface, the fabric having flexibility, strength and porosity effective for use as a hemostat; and further having a porous, polymeric matrix substantially homogeneously distributed on the first and second surfaces and through the fabric, the porous, polymeric matrix being made of a biocompatible, water-soluble or water-swellable cellulose polymer, wherein prior to distribution of the polymeric matrix on and through the fabric, the fabric contains about 3 percent by weight or more of water-soluble oligosaccharides.

U.S. Pat. No. 7,887,477 entitled “Method of improving cardiac function using a porous membrane” discloses a method whereby a porous membrane is inserted into a ventricle of a heart. The porous membrane creates a relatively hemostatic volume in which a thrombus can grow. Blood can still pass through fenestrations of the membrane into and out of the hemostatic volume. The fenestrations reduce pressures that act on the membrane, and so reduce stresses within the membrane. The flow characteristics through the hemostatic volume promote growth of the thrombus from a base of the hemostatic volume. The thrombus grows to slightly larger than the original size of the hemostatic volume so as to provide support for the membrane. Any remaining stresses within the membrane are thereby substantially eliminated. The thrombus shrinks over an ensuing period of time, with the membrane merely acting as a barrier to which an outer wall of the myocardium retracts. The function of the membrane is then complete, and may be absorbed.

U.S. Published Patent Application 2011/0070288 A1 entitled COMPOSITE LAYERED HEMOSTASIS DEVICE discloses a hemostatic composite structure having a bioabsorbable fabric or non-woven substrate having at least two major oppositely facing surface areas and a continuous non-porous polymer-based film that is laminated on one major surface of said substrate. The bioabsorbable fabric substrate can be an oxidized polysaccharide and/or the non-woven substrate can be made from bioabsorbable, non-cellulosic derived polymers. The continuous non-porous polymer based film can be a bioabsorbable polymer.

An article entitled “Localized Fluid Collection after Carrier-Bound Fibrin Sealant Application on Liver: Complication or Proof of Efficacy A Long-Term Clinical Observational Study” by Isidoro Di Carlo et al, published in Hepato-Gastroenterology 2011; 58:937-942, reported on the collection of fluids between collagen sponges and liver transaction surfaces. The observations include a statement that a collagen sponge that includes thrombin and fibrinogen when placed in contact with a wound surface is air-tight and fluid-tight, page 940, and that the tightness and adhesive strength of such a sponge should be considered a positive result. Page 941 conclusion.

In the instances of moderate and severe bleeding, there is a need to improve hemostatic patches because the performance is affected by the blood pooling under the patch and preventing good adhesion of the patch to the tissue.

### SUMMARY OF THE INVENTION

The present invention is directed in one embodiment to a hemostatic device having a patch that is made of a bioresorbable tissue compatible material. The patch has a tissue-facing surface and a top surface and at least one aperture or slit that penetrates said patch at least partially from said tissue-facing surface to said top surface. The device can be provided with an optional hemostatic agent that is either disposed on said tissue-facing surface or dispersed throughout said patch. The aperture or slit can be substantially round, having diameter from about 0.2 mm to about 5 mm; substantially elliptical, having length from about 2 mm to about 10 mm and width from about 0.2 mm to about 9 mm; or substantially rectangular, having length from about 2 mm to about 30 mm and width from about 0.01 mm to about 2 mm. The hemostatic agent can promote the rate of blood clotting, coagulation, or clotting and coagulation.

In one embodiment, the patch is a material that is selected from the group consisting of collagen, calcium alginate, chitin, polyester, polypropylene, polysaccharides, polyacrylic acids, polymethacrylic acids, polyamines, polyimines, polyamides, polyethers, polynucleotides, polynucleic acids, polypeptides, proteins, poly(alkylene oxide), polyalkylenes, polythioesters, polythioethers, polyvinyls, polymers comprising lipids, and mixtures thereof. In a preferred embodiment, the patch is composed primarily of collagen such that the primary scaffold material is a collagen material.

In another embodiment, the hemostatic device comprises a plurality of apertures or slits or combinations thereof. At least some of the plurality of apertures or slits or combinations thereof are structured and designed to open only when subjected to pressure, typically as a result of underlying or entrapped blood pressure that forces the device to rise, expand or flex which then results in an opening appearing or enlarging through which the previously entrapped blood can flow.

In another embodiment, the plurality of apertures or slits or combinations thereof can be distributed uniformly throughout the surface area of the patch. Alternatively, the plurality of apertures or slits or combinations thereof can be concentrated in a central region of the patch relative to the proportion of apertures, slits or combinations thereof that are provided in a perimeter or edge region of the patch. In a further alternative, the plurality of apertures or slits or combinations thereof can have a variety of sizes and shapes relative to one another herein wherein at least some of the relatively large size apertures or slits or combinations thereof are in a central portion of the patch. In another embodiment, the plurality of apertures or slits or combinations thereof can have larger openings on the wound-facing surface of the patch relative to the size of the corresponding openings on the top surface of the patch.

In a still further embodiment, the hemostatic device can have a multi-layer construction of a first patch layer and a second patch layer, wherein each patch layer has a plurality of apertures or slits or combinations thereof. The plurality of apertures or slits or combinations thereof in the first patch layer can either be fully aligned or not in complete alignment with the plurality of apertures or slits or combinations thereof in the second patch layer. The multi-layer construction is characterized by one or more distinct and separate layers that are affixed or otherwise attached to one another.

In one embodiment, the hemostatic device can be provided with at least one hemostatic agent that is derived from plasma. The at least one hemostatic agent can be provided on substantially all of the wound facing surface of the device or just a portion of the surface. Alternatively, the at least one hemostatic agent can be dispersed substantially throughout one or more patch layers. Still further, the at least one hemostatic agent can be provided as part of a separate carrier layer in a multilayer construct as described herein. The at least one hemostatic agent can be derived from human blood plasma.

In one embodiment, the hemostatic device is provided with at least one aperture or slit that does not extend entirely through the patch and runs parallel between the top surface and the wound-facing surface, preferably the at least one aperture or slit or combinations thereof runs for the entire width of the patch. In other words, the at least one aperture or slot or combinations thereof run start from one edge and can return to the same edge or to a different edge of the patch. In a still further embodiment, the at least one aperture or slit or combinations thereof runs from a central region of the patch to at least one edge of the patch.

The present invention is also directed to a method of use of a hemostatic device, comprising the steps of: providing a patch made of a bio-resorbable tissue compatible material, said patch having a tissue-facing surface and a top surface; providing an optional hemostatic agent disposed on said tissue-facing surface and optionally dispersed throughout said patch; and at least one aperture or slit penetrating said patch at least partially from said tissue-facing surface to said top surface, and applying said patch to a bleeding tissue or to a bleeding wound.

The present invention is also directed to a method of manufacturing of a hemostatic device, comprising the steps of: providing a patch made of a bio-resorbable tissue compatible material, said patch having a tissue-facing surface and a top surface; providing an optional hemostatic agent disposed on said tissue-facing surface and optionally dispersed throughout said patch, perforating said patch with at least one aperture or slit that penetrates said patch at least partially from said tissue-facing surface to said top surface.

### BRIEF DESCRIPTION OF THE FIGURES

FIGS. 1 through 13 show embodiments of the inventive fenestrated hemostat

### DETAILED DESCRIPTION OF THE INVENTION

In one embodiment, the present invention is directed towards a hemostat, comprising: a patch made of a bio-resorbable tissue compatible material, said patch having a tissue-facing surface and a top surface; an optional clotting and/or coagulation promoting agent disposed on said tissue-facing surface and optionally dispersed throughout said patch, and a plurality of apertures or slits, penetrating said patch at least partially from said tissue-facing surface to said top surface.

In another embodiment the present invention is directed towards a method of use of a hemostat, comprising the steps of: providing a patch made of a bio-resorbable tissue compatible material, said patch having a tissue-facing surface and a top surface; providing an optional clotting and/or coagulation promoting agent disposed on said tissue-facing surface and optionally dispersed throughout said patch, perforating said patch with a plurality of apertures or slits penetrating said patch at least partially from said tissue-facing surface to said top surface, and applying said patch to a bleeding tissue or to a bleeding wound.

In yet another embodiment the present invention is directed towards a method of manufacturing of a hemostat, comprising the steps of: providing a patch made of a bio-resorbable tissue compatible material, said patch having a tissue-facing surface and a top surface; providing an optional clotting and/or coagulation promoting agent disposed on said tissue-facing surface and optionally dispersed throughout said patch, perforating said patch with a plurality of apertures or slits penetrating said patch at least partially from said tissue-facing surface to said top surface.

The inventors have unexpectedly discovered that fenestrations such as apertures or slits in the hemostatic patch or pad are significantly improving performance of the patch when the bleeding rate is moderate or high. The fenestrations provided for the release of the blood pooling under the patch and allowed enough time for the clotting and sealing to occur at the interface with tissue.

Applicants discovered that a certain fenestrated or apertured hemostatic patch structure described more fully below that utilizes a fabric or non-woven material as a substrate, possesses improved properties suitable for use as a hemostat. The hemostatic structure of the present invention provides and maintains effective hemostasis when applied to a wound requiring hemostasis. Effective hemostasis, as used herein, is the ability to control and/or abate capillary, venous, or arteriole bleeding within an effective time, as recognized by those skilled in the art of hemostasis.

The hemostatic structure described below provides improved hemostasis, meaning decreasing the time to achieve hemostasis, which has great clinical significance. It will be shown that the present invention provides much improved hemostasis properties over conventional hemostats.

The hemostatic structure described below exhibit greater propensity and/or ability to stay in place during surgical procedures relative to existing hemostatic devices.

Substrate as used herein refers to the component of the hemostatic composite structure which is in direct contact to the wound surface. The substrates utilized in the present invention may be fabric/woven or nonwoven that provides form and shape and mechanical reinforcement necessary for use in hemostatic composite structures. In addition, the substrates are made of materials having hemostatic properties and be bioabsorbable. Bioabsorbable, biodegradable and bioresorbable as used herein refer to a material that is readily broken down internally by the mammalian body or broken down into components, which are consumed or eliminated in such a manner as not to interfere significantly with wound healing and/or tissue regeneration, and without causing any significant metabolic disturbance.

Polymers useful in preparing the fabric or non-woven substrates in hemostatic composite structure of the present invention include, without limitation, collagen, calcium alginate, chitin, polyester, polypropylene, polysaccharides, polyacrylic acids, polymethacrylic acids, polyamines, polyimines, polyamides, polyesters, polyethers, polynucleotides, polynucleic acids, polypeptides, proteins, poly(alkylene oxide), polyalkylenes, polythioesters, polythioethers, polyvinyls, polymers comprising lipids, and mixtures thereof.

In certain embodiments, wound dressings of the present invention are effective in providing and maintaining hemostasis in cases of severe bleeding. As used herein, severe bleeding is meant to include those cases of bleeding where a relatively high volume of blood is lost at a relatively high rate. Examples of severe bleeding include, without limitation, bleeding due to arterial puncture, liver resection, blunt liver trauma, blunt spleen trauma, aortic aneurysm, bleeding from patients with over-anticoagulation, or bleeding from patients with coagulopathies, such as hemophilia. Such wound dressings allow a patient to ambulate quicker than the current standard of care following, e.g. a diagnostic or interventional endovascular procedure.

The fabric substrates utilized in the present invention may be woven or nonwoven, provided that the fabric possesses the physical properties necessary for use in hemostatic wound dressings. A preferred woven fabric has a dense, knitted structure that provides form and shape for the hemostatic wound dressings. Such fabrics are described in U.S. Pat. No. 4,626,253, U.S. Pat. No. 5,002,551 and U.S. Pat. No. 5,007,916, the contents of which are hereby incorporated by reference herein as if set forth in its entirety.

The nonwoven substrates may be produced by melt-blown, electrospinning, needle punched methods and they can be preferably made from absorbable polymers. More specifically, absorbable nonwoven fabric is comprised of fibers that are not derived from cellulosic materials, such as comprising aliphatic polyester polymers, copolymers, or blends thereof, or comprised of gelatin, collagen, chitosan, and similar natural polymers, in various shapes, including, but not limited to, tapes, sponges, felts, and sheets. The aliphatic polyesters are typically synthesized in a ring opening polymerization of monomers including, but not limited to, lactic acid, lactide (including L-, D-, meso and D, L mixtures), glycolic acid, glycolide, E-caprolactone, p-dioxanone (1,4-dioxan-2-one), and trimethylene carbonate (1,3-dioxan-2-one). Examples of non-woven substrates are described in published U.S. Patent Application No. 2009/0104276 and published U.S. Patent Application No. 2006/0258995, the contents of which are hereby incorporated by reference herein as if set forth in their entireties. Other methods known for the production of nonwoven fabrics may be utilized and include such processes as air laying, wet forming and stitch bonding.

Other fabric constructions which produce equivalent physical properties may, of course, be utilized in the manufacture of the improved fabric or non-woven substrate and hemostatic composite structure of the present invention, and such constructions will be apparent to those skilled in the art. In a particularly preferred embodiment, the patch is constructed of one or more materials that are effectively non-porous such that blood and/or plasma becomes entrapped when the hemostatic device is applied onto a wound or bleeding tissue site. Such entrapped blood can cause the hemostatic device to expand, stretch, flex and/or bulge as result of continued blood flow.

Hemostatic agents that may be used in hemostatic composite structure according to the present invention include, without limitation, procoagulant enzymes, proteins and peptides, can be naturally occurring, recombinant, or synthetic, and may be selected from the group consisting of prothrombin, thrombin, fibrinogen, fibrin, fibronectin, heparinase, Factor X/Xa, Factor VII/VIIa, Factor IX/IXa, Factor XI/XIa, Factor XII/XIIa, tissue factor, batroxobin, ancrod, ecarin, von Willebrand Factor, collagen, elastin, albumin, gelatin, platelet surface glycoproteins, vasopressin and vasopressin analogs, epinephrine, selectin, procoagulant venom, plasminogen activator inhibitor, platelet activating agents, synthetic peptides having hemostatic activity, derivatives of the above and any combination thereof. Preferred hemostatic agents used in the present invention are thrombin, fibrinogen and fibrin.

Other therapeutic agents can be combined with the agents listed above to act in concert to facilitate other functions beyond hemostasis. Such agents include, but are not limited to, antibiotics, antimicrobials, anti-inflammatory agents, anti-neoplastic agents, and combinations thereof.

Such hemostatic composite structure of the present invention comprises hemostatic agents, including but not limited to thrombin, fibrinogen or fibrin, in an amount effective to provide rapid hemostasis and maintain effective hemostasis in cases of severe bleeding. If the concentration of the hemostatic agent in the wound dressing is too low, the hemostatic agent does not provide an effective procoagulant activity to promote rapid clot formation upon contact with blood or blood plasma. The agents may be incorporated into either the substrate or film components.

The hemostatic structure described herein may be used as an adjunct to primary wound closure devices, such as arterial closure devices, staples, and sutures, to seal potential leaks of gasses, liquids, or solids as well as to provide hemostasis. For example, the hemostat may be utilized to seal air from tissue or fluids from organs and tissues, including but not limited to, bile, lymph, cerebrospinal fluids, gastrointestinal fluids, interstitial fluids and urine. The laminated hemostasis device described herein has additional medical applications and may be used for a variety of clinical functions, including but not limited to tissue reinforcement and buttressing, i.e., for gastrointestinal or vascular anastomoses, approximation, i.e., to connect anastomoses that are difficult to perform (i.e. under tension), and tension releasing. The hemostat may additionally promote and possibly enhance the natural tissue healing process in all the above events. The hemostat can be used internally in many types of surgery, including, but not limited to, cardiovascular, peripheral-vascular, cardio-thoracic, gynecological, neuro- and general surgery.

According to the present invention, the apertures or fenestrations are fully or partially perforating the patch forming channels between the top surface and the tissue facing surface. The apertures or fenestrations are round, triangular, rectangular, or of any geometric shape. In one embodiment the fenestrations are narrow slits through the patch, having straight or curved shape. In one embodiment the fenestrations are perpendicular to the surface of the patch, while in other embodiments the fenestrations are non-perpendicular and are formed under angle to the surface of the patch.

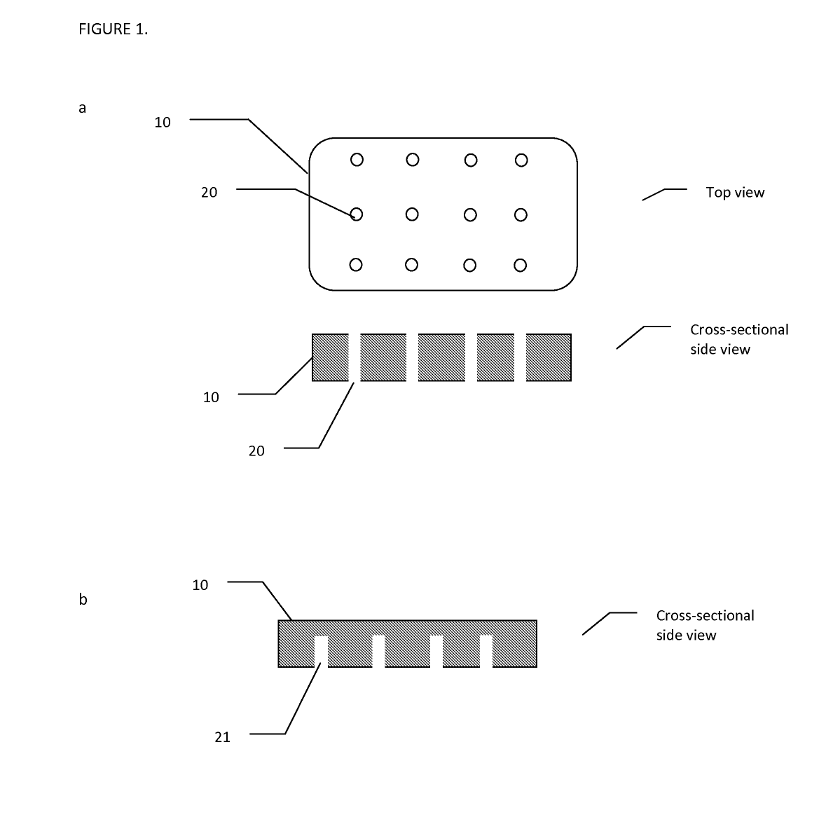

Referring now to FIG. 1, an embodiment of the fenestrated hemostat 10 with fenestrations or apertures 20 is shown in top view and cross-sectional side view, with apertures 20 having substantially uniform density throughout the body of the fenestrated hemostat 10. FIG. 1a shows fenestrations or apertures 20 fully penetrating the body of the fenestrated hemostat 10. FIG. 1b, in a cross-sectional side view, shows fenestrations or apertures 21 partially penetrating the body of the fenestrated hemostat 10.

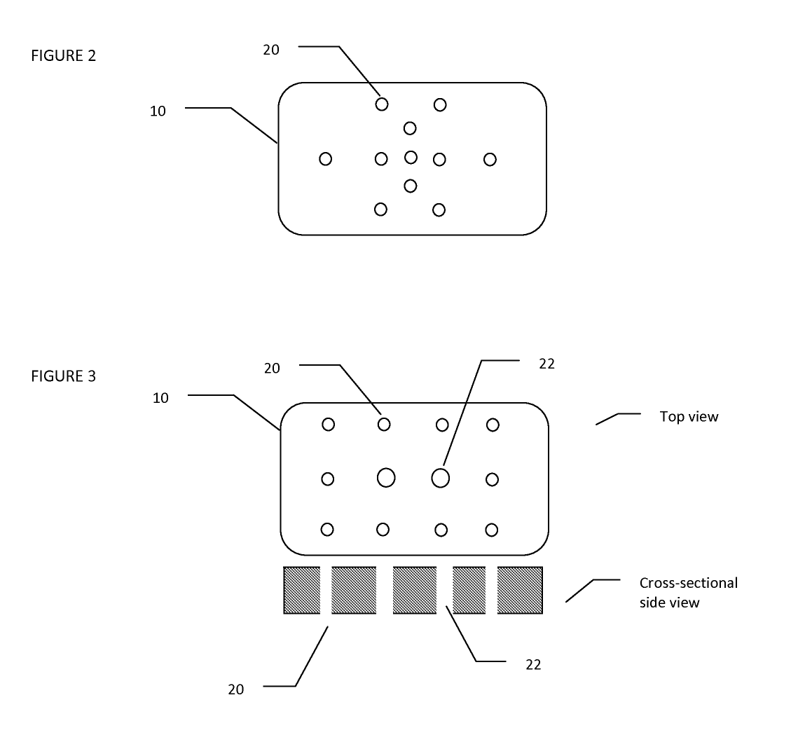

Referring now to FIG. 2, an embodiment of the fenestrated hemostat 10 with fenestrations or apertures 20 is shown in top view, with apertures 20 having higher density in the middle section of the fenestrated hemostat 10, resulting in faster blood pressure release in the middle section of fenestrated hemostat 10.

Referring now to FIG. 3, an embodiment of the fenestrated hemostat 10 with smaller apertures 20 and larger apertures 22 is shown in top view and cross-sectional side view, with larger apertures 22 positioned predominantly in the middle section of the fenestrated hemostat 10 and smaller apertures 20 positioned predominantly in the peripheral section of the fenestrated hemostat 10, resulting in faster blood pressure release in the middle section of fenestrated hemostat 10.

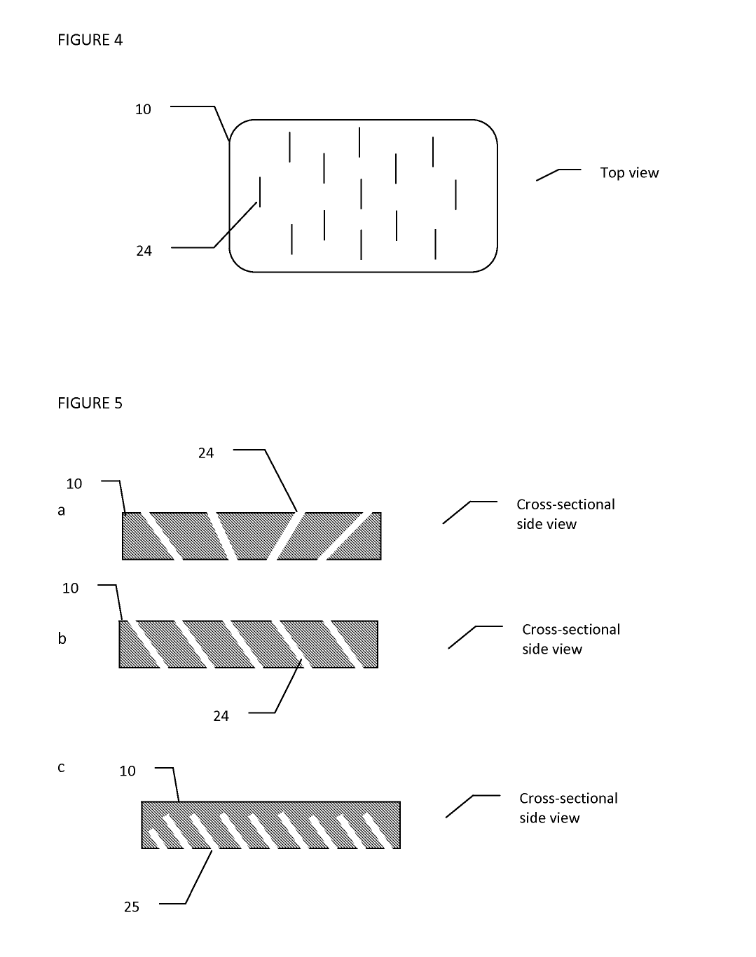

Referring now to FIG. 4, an embodiment of the fenestrated hemostat 10 with fenestrations or elongated slits 24 is shown in top view, with slits 24 having substantially uniform density throughout the body of the fenestrated hemostat 10. In an alternative embodiment (not shown), slits 24 are positioned with higher density or lower density in the middle section of the fenestrated hemostat 10, resulting in faster blood pressure release in the middle section of fenestrated hemostat 10.

Referring now to FIG. 5, an embodiment of the fenestrated hemostat 10 with fenestrations or elongated slits 24 is shown in cross-sectional side view, with slits 24 penetrating the body of the fenestrated hemostat 10 under angle, which can be the same angle as shown in FIG. 5b, or variable angle, such as shown in FIG. 5a. Fenestrations or slits 24 are non-perpendicular to the surface. Optionally exerting pressure on surface of fenestrated hemostat 10 will result in closing of fenestrations or slits 24. As shown in FIGS. 5a and 5b, slits 24 are fully penetrating the body of the fenestrated hemostat 10. FIG. 5c shows, in a cross-sectional side view, that slits 25 are partially penetrating the body of the fenestrated hemostat 10.

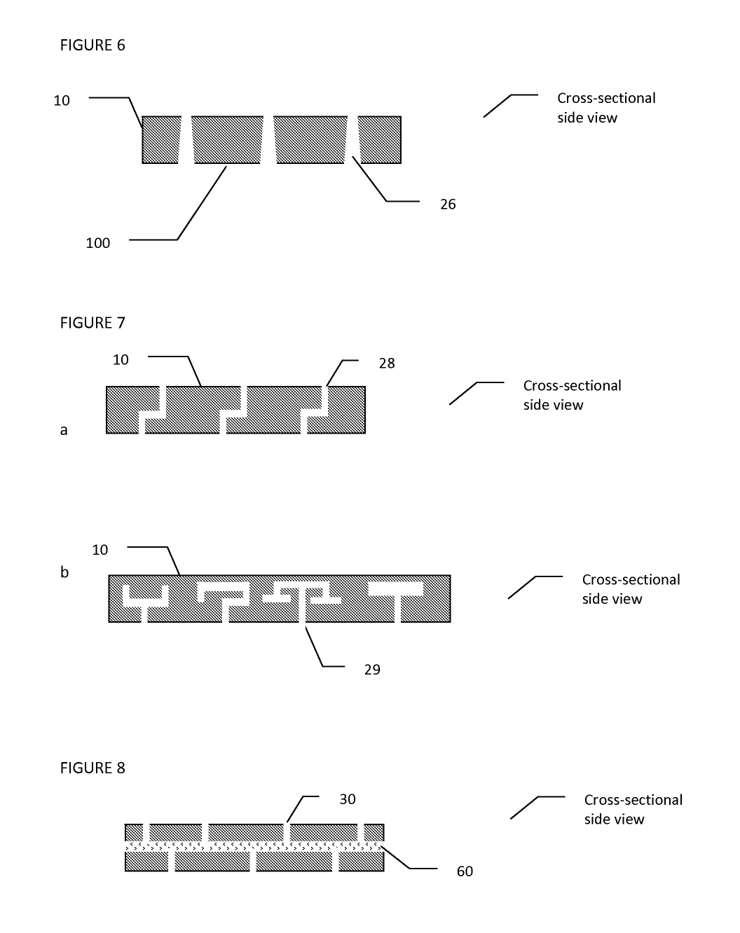

Referring now to FIG. 6, an embodiment of the fenestrated hemostat 10 with fenestrations or conical apertures 26 is shown in cross-sectional side view; with conical apertures 26 having conical shape and penetrating body of fenestrated hemostat 10 with wider or larger opening of conical aperture 26 on the wound facing surface 100 of fenestrated hemostat 10.

Referring now to FIG. 7a, an embodiment of fenestrated hemostat 10 with tortuous path fenestrations 28 is shown in cross-sectional side view. Tortuous path fenestrations 28 are penetrating body of fenestrated hemostat 10 via non-linear path. This embodiment can be manufactured, for example, by combining two or more fenestrated hemostatic pads, positioned one on top of another with offset fenestrations.

Referring now to FIG. 7b, an embodiment of fenestrated hemostat 10 with several different types of tortuous path fenestrations 29 is shown in cross-sectional side view. Tortuous path fenestrations 29 are penetrating body of fenestrated hemostat 10 only partially and via non-linear path, providing for higher capacity of absorbing blood or other fluids.

Referring now to FIG. 8, an embodiment of fenestrated hemostat 10 comprising a multi-layer pad with offset fenestrations 30 and optional porous interlayer 60 is shown in cross-sectional side view. This embodiment can be manufactured, for example, by combining two or more fenestrated hemostatic pads, positioned one on top of another with offset fenestrations 30 and optional porous interlayer 60 between two or more fenestrated hemostatic pads. Such sandwich arrangement can also be method of use, with at least two fenestrated pads layered one on top of another. The second layer can be lower cost component, containing less biologics or no biologics.

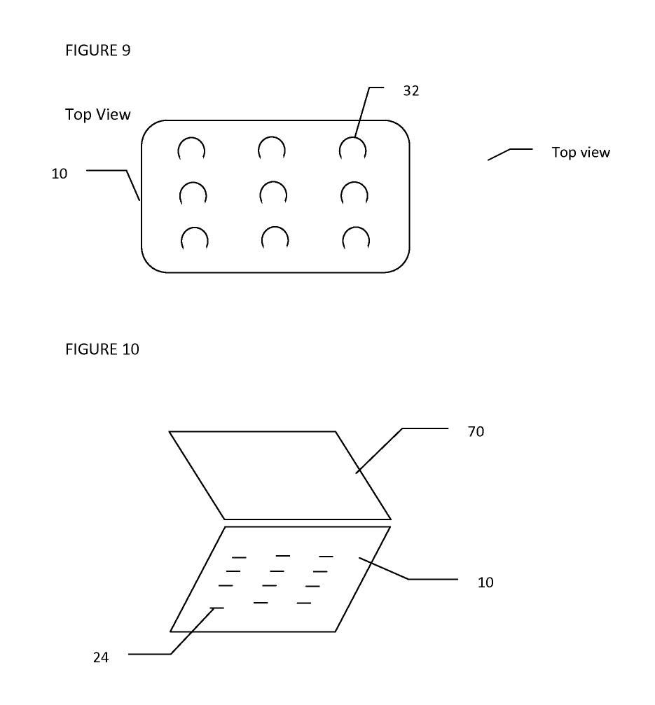

Referring now to FIG. 9, an embodiment of fenestrated hemostat 10 having flaps 32 on the surface is shown in top view. Flaps 32 are adapted to release blood pooling under fenestrated hemostat 10 when the bleeding is moderate or severe but not releasing blood when bleeding rate is low or pressure build-up is not significant. Flaps 32 are adapted to lift up and release pressure and then close back onto surface of fenestrated hemostat 10.

Referring now to FIG. 10, an embodiment of fenestrated hemostat 10 having optional cover flap 70 and slits 24 is shown in 3D view. Cover flap 70 can be used to seal the surface and slits 24 of fenestrated hemostat 10 for low bleeding rates or keep slits 24 open for moderate and severe bleeding hemostasis. Cover flap 70 can also be optionally impregnated or coated with fibrinogen, thrombin, or both. Fenestrated hemostat 10 can also be optionally impregnated or coated with fibrinogen, thrombin, or both. Cover flap 70 can also be coated or impregnated with a clotting factor which is different from clotting factor used to coat or to impregnate body of fenestrated hemostat 10. In one embodiment cover flap 70 is coated or impregnated with thrombin, and body of fenestrated hemostat 10 is coated or impregnated with fibrinogen. In another embodiment cover flap 70 is coated or impregnated with fibrinogen, and body of fenestrated hemostat 10 is coated or impregnated with thrombin.

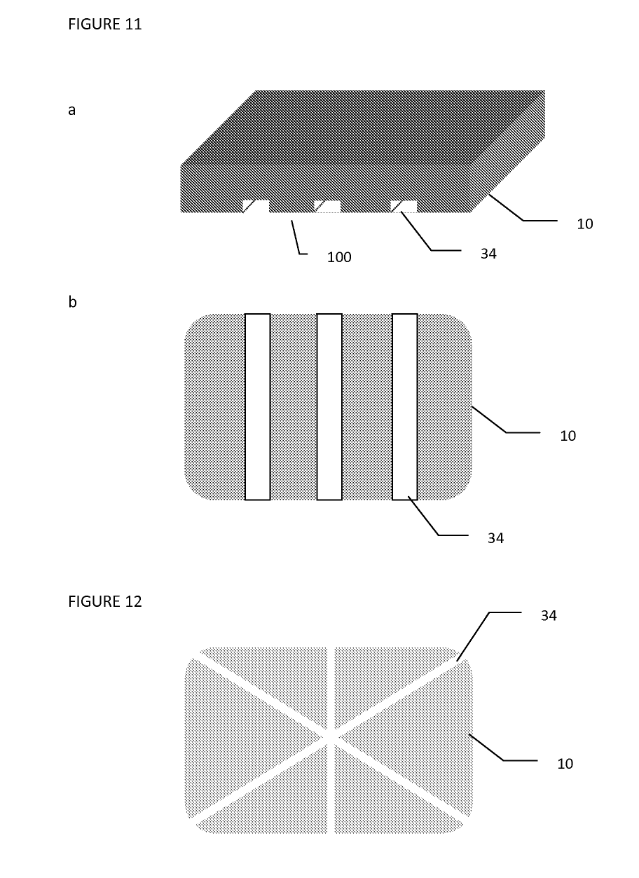

Referring now to FIG. 11, an embodiment of fenestrated hemostat 10 having substantially parallel channels 34 on wound facing surface 100 of fenestrated hemostat 10 is shown in 3D view on FIG. 11a and is shown in surface view from tissue-facing side 100 on FIG. 11b. Channels 34 are adapted to channel blood from underneath of the fenestrated hemostat 10.

Referring now to FIG. 12, an embodiment of fenestrated hemostat 10 having radially positioned channels 34 on wound facing surface 100 of fenestrated hemostat 10 is shown in surface view from tissue-facing side 100. Channels 34 are adapted to channel blood from underneath of the fenestrated hemostat 10.

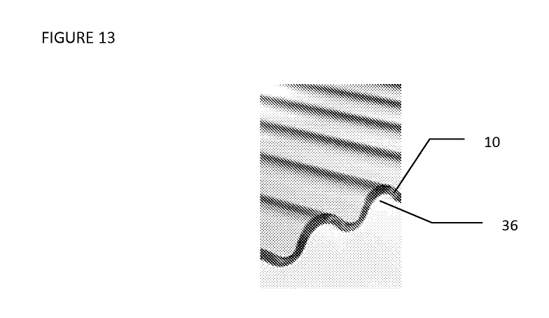

Referring now to FIG. 13, an embodiment of fenestrated hemostat 10 having corrugated surface forming channels 36 is shown 3D view. Channels 36 are adapted to channel blood from underneath of the fenestrated hemostat 10.

In one embodiment, the density of the apertures or fenestrations is uniform. In another embodiment, there are more apertures or fenestrations per sq. cm in the center of the hemostatic pad, and less apertures or fenestrations on the periphery of the hemostatic pad, resulting in faster blood pressure release in the middle section of fenestrated hemostat 10.

The dimensions of fenestrations are from about 0.05 mm² to about 50 mm², such as 1 or 3 or 10 mm². The total open area of apertures or fenestrations is from about 0.01% to about 25% of the overall total patch surface area, such as about 0.1% or 1.0% or 5% of the surface area of the patch.

In one embodiment the apertures or fenestrations are adapted to be closed when bleeding rate is low, and adapted to open to release blood through when the blood flow is moderate or severe or when pressure of blood pooling under the patch is increasing.

Optionally, clotting and/or coagulation promoting agents can be disposed on tissue-facing surface and optionally dispersed throughout the patch. Optionally, epinephrine or other active agents can be disposed on the tissue facing side of the patch. The patch is preferably made of natural or synthetic bio-resorbable material. In one embodiment, an addition of fibrinolytic inhibitors is contemplated. It is contemplated that there is potential for interaction of the blood with biologics along the fenestrations or channels plus physical constituents of the fenestrations channels (collagen, cotton) will provide increased surface area for interaction plus physical anchoring.

In one embodiment the device is pleated so that its apparent surface area is increased at the time of application, and space is afforded to take up excess blood.

In one embodiment the inventive hemostat is applied to tissue surface or to non-bleeding surface, prior to making surgical incision.

While the following examples demonstrate certain embodiments of the invention, they are not to be interpreted as limiting the scope of the invention, but rather as contributing to a complete description of the invention.

### EXAMPLE 1

TachoSil® medicated sponge (available, for example from Nycomed, Linz, Austria) was utilized in experimental testing of the present invention. TachoSil® is an equine-derived collagen pad coated with a dry layer of human fibrinogen and human thrombin at a mean concentration of 5.5 mg and 2.0 units, respectively. The coated side of the pad also contains riboflavin, which imparts a yellow color and indicates the side to be placed against the wound.

TachoSil® is a fixed combination of human fibrinogen and human thrombin on a solid equine collagen patch. The active side is colored yellow using riboflavin. As soon as the active side comes into contact with liquid (such as blood) the fibrinogen and thrombin dissolve and interact, causing the formation of fibrin monomers. These polymerize to fibrin polymers, which are subsequently cross linked forming a fibrin clot. The fibrin clot causes the collagen sponge to adhere to the wound surface and thus provides sealing properties. TachoSil® contains per square centimeter 5.5 mg human fibrinogen and 2.0 I.U. human thrombin, thus one large size patch (9.5 cm×4.8 cm×0.5 cm) contains approximately 250 mg human fibrinogen and 90 IU human thrombin.

TachoSil Ex-Vivo Heparinized Aorta Model

The hemostatic performance of a collagen-based medicated sponge (Tachosil™) was evaluated in an ex-vivo bench top circulatory cardiopulmonary bypass (CPB) model. Fresh porcine blood, obtained from an intravenous catheter draw from a donor animal, was anticoagulated with heparin and used in the study.

The bench top cardiopulmonary bypass consisted of a silicone tubing flow loop with an integrated oxygentator/cooler section and roller pump to establish the proper pulse rate and blood pressure. The heparinized porcine blood was recirculated through the flow loop circuit. The heparinized blood was titrated with protamine to partially reverse the initial heparin dose and maintain an Activated Clotting Time (ACT) of approximately 400-500 sec. The blood was oxygenated to 100% saturation, warmed to 37° C., and blood pressure of 120/80 mmHg was established at the test site.

A section of the silicone tubing flow loop was replaced with a collagen-coated vascular graft (Hemashield™) that served as the test section.

The hemostatic performance of the Tachosil™ collagen pad was assessed using standardized defects on the Hemashield™ graft test section with heparinized blood anticoagulated to ACT levels between 400-500 sec. The level of blood anticoagulation was analogous with that seen in the clinical scenario of high anticoagulation. A full wall linear defect, approximately 5-6 mm in length, was created in the Hemashield™ graft using a number 11-Blade scalpel. A 1″×1″ piece of Tachosil™ collagen pad was moistened with saline and applied over the bleeding site using moist gauze as per the manufacturer's Instructions for Use. The Tachosil™ collagen pad was held in place with sufficient pressure to prevent continued bleeding for a period of three minutes.

After the three-minute tamponade period, the gauze was gently removed and the Tachosil™ collagen pad began to lose its adherence around the defect edges. The blood-induced stress applied to the collagen pad/graft interface resulted in loss of adherence of the pad and ultimately re-bleeding from one or more the edges. Several applications of the product produced similar and consistent results.

A modification was made to the dry Tachosil™ collagen medicated sponge by creating an array of linear full wall thickness defects (fenestrations) in the collagen pad.

A 1″×1″ piece of dry Tachosil™ collagen pad was placed on a soft surface (several gauze pads) with the collagen side facing up. Multiple full wall linear incisions were created using a number 11-blade scalpel by passing the knife blade through the pad at a 45° angle creating three rows of four liner defects each measuring about 3-4 mm in length on the collagen pad surface. The modified collagen pad was applied to a similar defect in the same manner as the non-modified pad for three minutes. After the tamponade period, the moist gauze pad was removed and the site was hemostatic with good adhesion of the Tachosil™ collagen pad. A few droplets of blood that were slowly escaping through some of the fenestrations soon clotted off.

The above experimental observations demonstrate that fenestrations in the matrix or substrate of the hemostatic pad, unexpectedly, improved the hemostatic performance while providing some initial migration of blood through the pad, and reduced the blood-pad interface pressure induced stress levels. Without wishing to be bound by theory, the inventors conclude that the reduced stress at the blood-pad interface precluded the hemostasis pad from being “pushed off” the wound site, which would result in an adhesive failure. Without fenestrations, The Tachosil™ matrix, which is a closed-cell equine collagen-based product, creates a thin monolithic layer on the wound site and does not allow blood to flow into the matrix, which results in increased pressure stress at the interface. The increased stress level at the hemostat tissue interface, as the material peeled away, resulted in complete delamination from the wound site and re-bleeding.

The fenestrations improved the hemostatic performance, adhesion, and sealing properties of the hemostatic pad. The application of the modified (fenestrated) product was repeated several times, over different but comparable defects, with similar observations.

1-17. (canceled)

18. A method of use of a hemostatic device, comprising the steps of:
providing a non-porous patch made of a bio-resorbable tissue compatible material, said patch having a tissue-facing surface and a top surface;
providing an optional hemostatic agent that is disposed on said tissue-facing surface or optionally dispersed throughout said patch or optionally as a separate carrier layer, said patch having at least one aperture or slit or combinations thereof that penetrate said patch at least partially from said tissue-facing surface to said top surface, and applying said patch to a bleeding tissue or to a bleeding wound.

19. (canceled)
